(19) United States
(12) Patent Application Publication
(10) Pub. No.: US 2010/0204561 A1
(43) Pub. Date: Aug. 12, 2010

(54) IMAGING CATHETERS HAVING IRRIGATION

(75) Inventors: Vahid SAADAT, Atherton, CA (US); Zachary J. MALCHANO, San Francisco, CA (US); Veerappan SWAMINATHAN, Sunnyvale, CA (US)

Correspondence Address: LEVINE BAGADE HAN LLP, 2400 GENG ROAD, SUITE 120, PALO ALTO, CA, 94303, US

(73) Assignee: Voyage Medical, Inc., Redwood City, CA (US)

(21) Appl. No.: 12/703,997

(22) Filed: Feb. 11, 2010

Related U.S. Application Data
(60) Provisional application No. 61/151,764, filed on Feb. 11, 2009.

(51) Int. Cl. A61B 18/18; A61B 5/05

(52) U.S. Cl. 600/407; 606/15

(57) ABSTRACT

Imaging catheters having irrigation capabilities are described herein. Generally, the device may include a first inner membrane which is sealed and serves to position the device within or relative to a lumen. This balloon structure, when filled with fluid, expands and is engaged in direct contact with the tissue. A second (outer) membrane is not completely sealed and instead provides a pathway for delivery of fluid at the treatment site for effecting various treatments. Imaging systems, optionally articulatable, may be positioned within the balloon as well as electrodes positionable upon the balloon may be utilized to facilitate tissue treatments.

### CROSS-REFERENCE TO RELATED APPLICATIONS

This application claims priority to U.S. Provisional App. 61/151,764 filed Feb. 11, 2009, which is incorporated herein by reference in its entirety.

### FIELD OF THE INVENTION

The present invention relates generally to balloon catheter devices used for imaging and treating tissue regions of interest. More particularly, the present invention relates generally to methods and devices for balloon catheters which can be used to image and treat tissue regions of interest, such as a vessel ostium, for various conditions, such as atrial fibrillation, etc.

### SUMMARY OF THE INVENTION

An inflatable balloon catheter having an irrigation sheath may generally comprise two expandable membranes disposed about a catheter. The first inner membrane may be generally or substantially sealed to a catheter and may serve as a balloon to facilitate positioning of the device, e.g., within a lumen. This balloon structure when filled with fluid may expand and become engaged in direct contact with the tissue. A second (outer) membrane may be at least partially positioned over the balloon and may provide a pathway for delivery of fluid at the treatment site. In treating a tissue region with ablation energy, particularly within a body lumen such as a heart chamber, one device in particular may be used as shown and described in detail in U.S. Pat. No. 6,605,055, which is incorporated herein by reference in its entirety. As disclosed, an inflatable balloon which is sealed to a catheter may be advanced within a body lumen, such as within a chamber of a subject's heart, and inflated for contact against a tissue region to be treated.

A balloon catheter having a primary balloon member disposed about a catheter for inflation within the body, e.g., with the heart, may provide a transmission waveguide for radiation (such as laser radiation) projecting from an optical fiber to the ablation site, e.g., an ostium of a vessel. The catheter is typically an elongated hollow instrument having at least one lumen in communication with the port.

The outer membrane or sheath may define a distal opening to partially cover the primary balloon such that an irrigating fluid such as saline may be introduced through the annular conduit between the inner and outer membranes and exit through this opening to clear the region of blood between the balloon and the underlying tissue. In phototherapy applications, the removal of blood from the treatment site allows for the unobstructed and uniform delivery of ablative energy. In addition, the irrigating fluid cools the surface of the target site, thereby preventing overheating or burning of the tissue or coagulation. Also, it is noted that removal of blood allows direct visualization of the tissue surface with an appropriate imaging system.

An imager, e.g., CMOS or CCD electronic image sensor, may be affixed to an inside wall of the primary balloon with an electrical connection leading out of the distal end of the balloon to an image processing system for displaying the image, e.g., on a monitor. Direct visualization of the tissue surface is made possible when blood is flushed out and/or squeezed from the field of view. At least one light source, such as an LED, may also be affixed to an inside wall of the primary balloon coupled to an electrical connection as well. Both the light source and imager may be angled or positioned such that their field of view is directed towards the distal end of the balloon to capture and/or illuminate the underlying tissue region through the transparent balloon.

Another variation may include a fiberscope, which may be articulatable to control a direction of its distal end, positioned within the interior of the balloon. The distal end of the fiberscope may be articulated from outside the patient's body by the operator to direct an angle of the fiberscope within the balloon to view any region of contacted tissue through the balloon. The fiberscope may be optionally coupled to an imaging system, e.g., CMOS or CCD electronic image sensor, positioned external to the patient's body.

In yet another variation, an imager, such as an electronic imager, may be positioned upon the distal end of an articulatable member. The articulatable member may be manipulated from outside the patient's body to direct a viewing angle of the imager within the balloon. An imaging system may be located outside the patient's body for communicating with the imager for processing and/or displaying the images of the contacted tissue regions captured within the patient.

In yet another variation, fluid such as saline may be introduced through the conduit formed between the sheath and balloon. The introduced fluid, particularly an electrolytic fluid such as saline, may also be used to conduct ablative energy into the underlying tissue from the one or more electrodes which may be positioned along an outer surface of the balloon or sheath. The one or more electrodes may be positioned at locations where the fluid exits the conduit and contacts the underlying tissue such that the fluid flowing into contact with the electrodes may conduct any discharged energy, e.g., radio frequency (RF) energy, to ablate the tissue in combination with or exclusive of the ablative radiation energy projected from the optical fiber. The energy delivered via electrodes is not limited to RF energy may but also include any number of other ablative forms of energy such as cryo-ablation, microwave, ultrasonic, etc. Moreover, utilization of ablation energy in contact or in direct proximity to the tissue may provide additional ablative effects should blood obscure the radiation energy. Also, these electrodes may be also used independently from the laser ablation system or may be used to map electric potentials at the tissue surface.

Another variation of the balloon catheter may include one or more pores defined circumferentially about the distal end of the sheath such that the introduced fluid passing through the conduit may be diffused through the one or more pores into contact against the underlying tissue. The diffusion of the fluid through the pores may facilitate distribution of the ablation energy over the tissue.

In yet another variation, one or more ultrasound transducers may be positioned near or at a distal end of the balloon and/or sheath for placement in proximity to or in contact against the tissue region of interest. The one or more ultrasound transducers may be actuated to deliver ultrasonic signals into the underlying tissue to detect a thickness of the tissue, e.g., at the locations at which the radiative energy is to be applied. Knowledge of the thickness of the tissue to be ablated may help determine how much energy to provide or to determine, e.g., an appropriate amount of fluid flow needed to cool the tissue surface, etc., amongst other parameters. Tissue thickness detection utilizing ultrasound transducers is described in further detail in U.S. patent application Ser. No. 12/118,439 filed May 9, 2008 (U.S. Pat. Pub. 2009/0030412 A1), which is incorporated herein by reference in its entirety. Various methods may be utilized, e.g., by a controller such as a microprocessor in communication with the ultrasound transducers, for controlling and/or adjusting various parameters of an ablation procedure, e.g., power, laser intensity, flow rate, temperature, etc.

### BRIEF DESCRIPTION OF THE DRAWINGS

FIG. 1 shows a cross-sectional view of a variation of a balloon catheter apparatus having an imaging system attached to an inside wall of the balloon.

FIG. 2 shows a cross-sectional view of another variation of a balloon catheter apparatus having an articulatable fiberscope within the balloon and coupled at a proximal end to an imaging system, such as a CMOS or CCD imaging system.

FIG. 3 shows a cross-sectional view of another variation of a balloon catheter apparatus having imaging sensor, such as an electronic imager, affixed at the distal end of an articulatable member within the balloon.

FIG. 4 shows a cross-sectional side view of yet another variation illustrating one or more electrodes positioned upon the balloon surface for transmitting energy through a fluid into the underlying tissue.

FIG. 5 shows a cross-sectional side view of a balloon catheter apparatus which defines a plurality of openings or pores through which energy may be transmitted into the underlying tissue surface.

FIG. 6 shows a cross-sectional side view of a balloon catheter apparatus having one or more ultrasound transducers positioned along the balloon for detecting a thickness of the underlying tissue.

FIG. 7 shows an example of a flow chart illustrating one method for controlling parameters of an ablation system in response to detected tissue thickness.

### DETAILED DESCRIPTION OF THE INVENTION

In treating a tissue region with ablation energy, particularly within a body lumen such as a heart chamber, various devices and methods may be utilized for visualizing and treating the tissue. One device is shown and described in detail in U.S. Pat. No. 6,605,055, which is incorporated herein by reference in its entirety. As disclosed, an inflatable balloon which is sealed to a catheter may be advanced within a body lumen, such as within a chamber of a subject's heart, and inflated for contact against a tissue region to be treated.

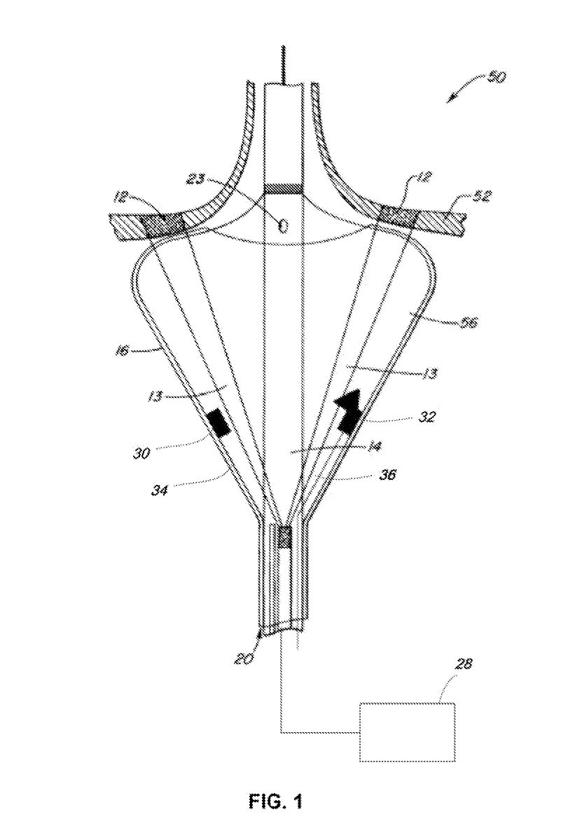

FIG. 1 illustrates a cross-sectional side view of a balloon catheter 50 having a primary balloon member 56 disposed about a catheter 14 for inflation (via port 23) within the body (e.g., with the heart) to provide a transmission waveguide for radiation 13 (such as laser radiation) projecting from an optical fiber to the ablation site 12, e.g., an ostium of a vessel. A laser generator 28 may be in optical communication with the optical fiber for delivering the radiation 13. The primary balloon member 56 may be generally or substantially sealed and can be inflated to position the catheter 14 within the lumen. The catheter 14 is typically an elongated hollow instrument having at least one lumen in communication with the port 23. The primary balloon 56 is shown engaged in direct contact with a body lumen 52 (e.g., a pulmonary vein) and an outer membrane or sheath 16 may be at least partially disposed about the primary balloon member 56 for providing an irrigation path via the annular conduit 20 formed between the two membranes to the body lumen. Primary balloon member 56 and sheath 16 may accordingly form a respective inner and outer membrane of the balloon assembly.

The outer membrane or sheath 16 may define a distal opening to partially cover the primary balloon 56, as shown, such that an irrigating fluid such as saline may be introduced through the annular conduit 20 between the inner and outer membranes and exit through this opening to clear the region of blood between the balloon and the underlying tissue. In phototherapy applications, the removal of blood from the treatment site allows for the unobstructed and uniform delivery of ablative energy 13. In addition, the irrigating fluid cools the surface of the target site, thereby preventing overheating or burning of the tissue or coagulation. Also, it is noted that removal of blood allows direct visualization of the tissue surface with an appropriate imaging system.

In this variation, an imager 32, e.g., CMOS or CCD electronic image sensor, may be affixed to an inside wall of the primary balloon 56 with an electrical connection 34 leading out of the distal end of the balloon to an image processing system for displaying the image, e.g., on a monitor. Direct visualization of the tissue surface is made possible when blood is flushed out and/or squeezed from the field of view. At least one light source 30, such as an LED, may also be affixed to an inside wall of primary balloon 56 coupled to an electrical connection 36 as well. Both light source 30 and imager 32 may be angled or positioned such that their field of view is directed towards the distal end of the balloon 56 to capture and/or illuminate the underlying tissue region 52 through the balloon 56 which may be optically transparent.

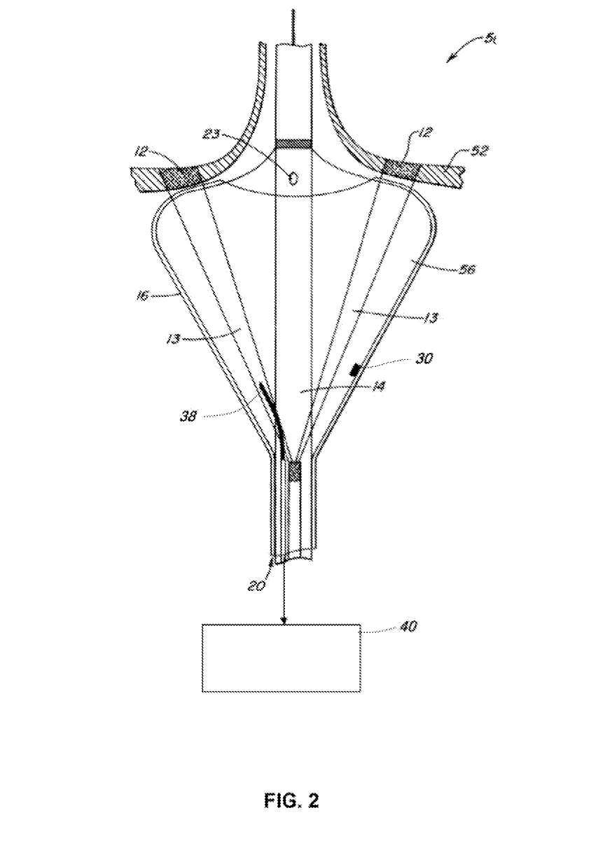

Another variation is illustrated in the cross-sectional side view of FIG. 2, which shows a fiberscope 38, which may be articulatable to control a direction of its distal end, positioned within the interior of balloon 56. The distal end of fiberscope 38 may be articulated from outside the patient's body by the operator to direct an angle of fiberscope 38 within the balloon 56 to view any region of contacted tissue through the balloon 56. The fiberscope 38 may be optionally coupled to an imaging system 40, e.g., CMOS or CCD electronic image sensor, positioned external to the patient's body.

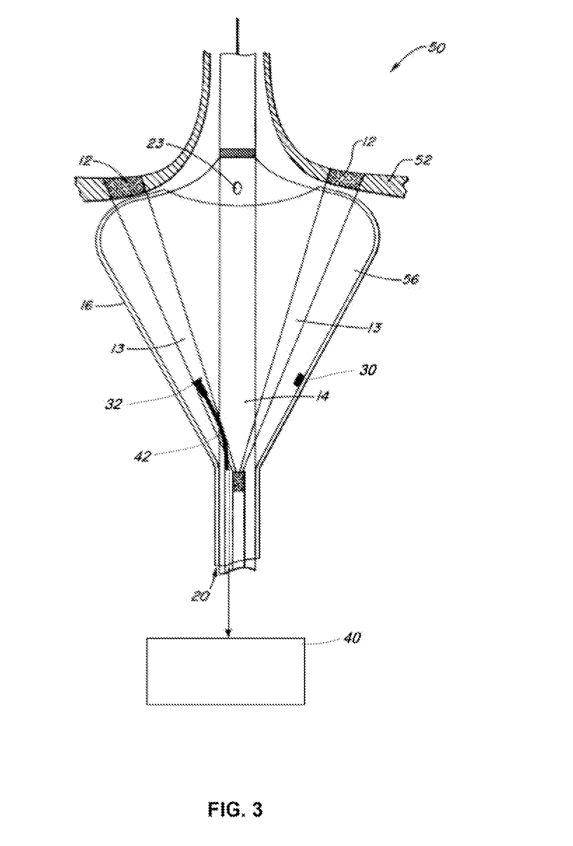

In yet another variation, FIG. 3 shows an example where an imager, such as an electronic imager 32, may be positioned upon the distal end of an articulatable member 42. As previously described, articulatable member 42 may be manipulated from outside the patient's body to direct a viewing angle of imager 32 within the balloon 56. Imaging system 40 may be located outside the patient's body for communicating with the imager 32 for processing and/or displaying the images of the contacted tissue regions captured within the patient.

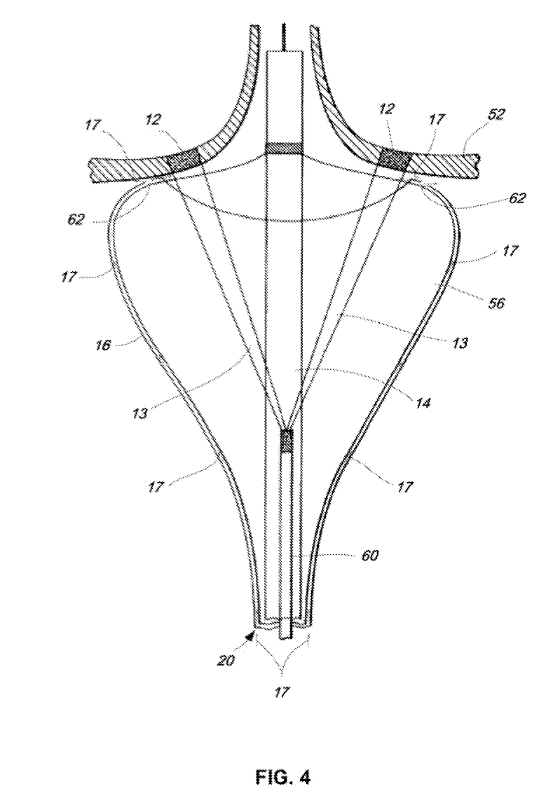

In yet another variation, FIG. 4 shows a cross-sectional side view of a tissue region in proximity to body lumen 52 ablated by projecting radiation 13 from optical fiber 60 positioned within balloon 56. As previously described, fluid 17 such as saline may be introduced through conduit 20 formed between sheath 16 and balloon 56. The introduced fluid 17, particularly an electrolytic fluid such as saline, may also be used to conduct ablative energy into the underlying tissue from one or more electrodes 62 which may be positioned along an outer surface of balloon 56 and/or sheath 16, e.g., near or at a distal end of the balloon 56 and/or sheath 16. The one or more electrodes 62 may be positioned at locations where fluid 17 exits conduit 20 and contacts the underlying tissue 52 such that the fluid 17 flowing into contact with electrodes 62 may conduct any discharged energy, e.g., radio frequency (RF) energy, to ablate the tissue 52 in combination with or exclusive of the ablative radiation energy 13 projected from optical fiber 60. The energy delivered via electrodes 62 is not limited to RF energy may but also include any number of other ablative forms of energy such as cryo-ablation, microwave, ultrasonic, etc. Moreover, utilization of ablation energy in contact or in direct proximity to the tissue may provide additional ablative effects should blood obscure the radiation energy 13. Also, these electrodes 62 may be also used independently from the laser ablation system or may be used to map electric potentials at the tissue surface.

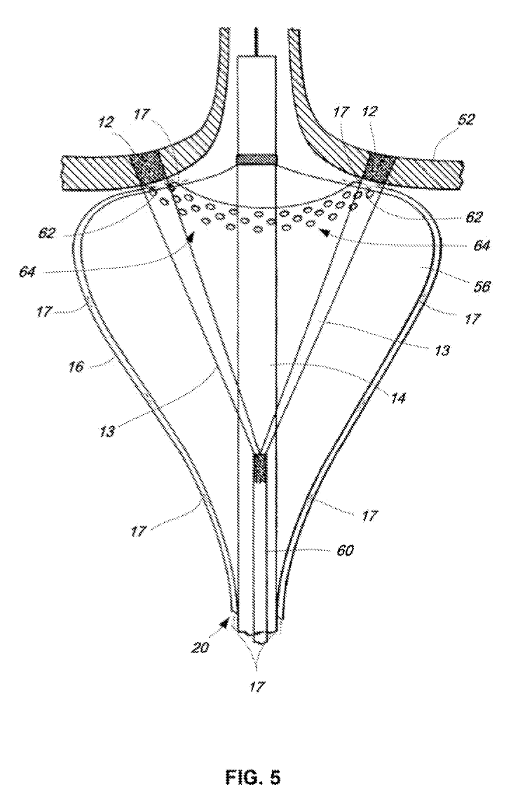

Another variation of balloon catheter 50 is shown in the cross-sectional side view of FIG. 5. This example illustrates a balloon catheter assembly similarly configured to the variation shown in FIG. 4 with one or more electrodes 62 for delivering ablation energy conducted via the discharged fluid 17. In this variation, however, one or more pores 64 may be defined circumferentially about the distal end of the sheath 16 such that the introduced fluid 17 passing through conduit 20 may be diffused through the one or more pores 64 into contact against the underlying tissue. The diffusion of the fluid 17 through the pores 64 may facilitate distribution of the ablation energy over the tissue.

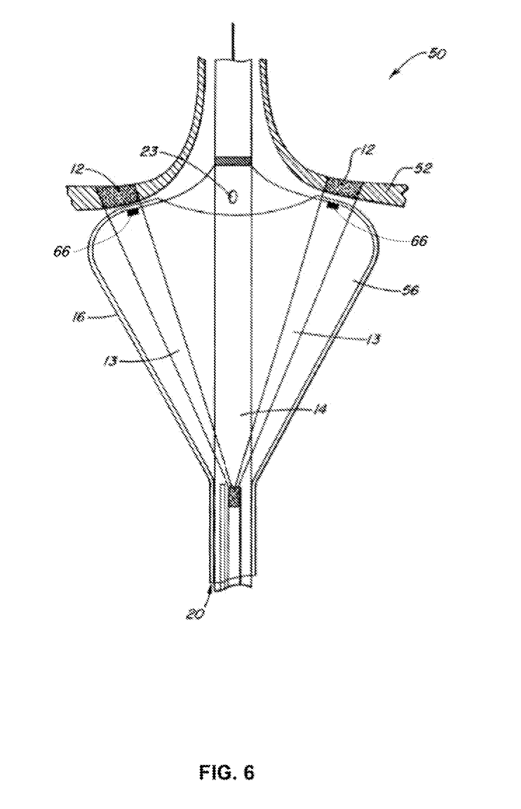

In yet another variation, FIG. 6 shows a cross-sectional side view of a balloon assembly having one or more ultrasound transducers 66 positioned near or at a distal end of the balloon 56 and/or sheath 16 for placement in proximity to or in contact against the tissue region of interest. The one or more ultrasound transducers 66 may be actuated to deliver ultrasonic signals into the underlying tissue to detect a thickness of the tissue, e.g., at the locations 12 at which the radiative energy 13 is to be applied. Knowledge of the thickness of the tissue to be ablated may help determine how much energy to provide or to determine, e.g., an appropriate amount of fluid flow needed to cool the tissue surface, etc., amongst other parameters. Tissue thickness detection utilizing ultrasound transducers is described in further detail in U.S. patent application Ser. No. 12/118,439 filed May 9, 2008 (U.S. Pat. Pub. 2009/0030412 A1), which is incorporated herein by reference in its entirety.

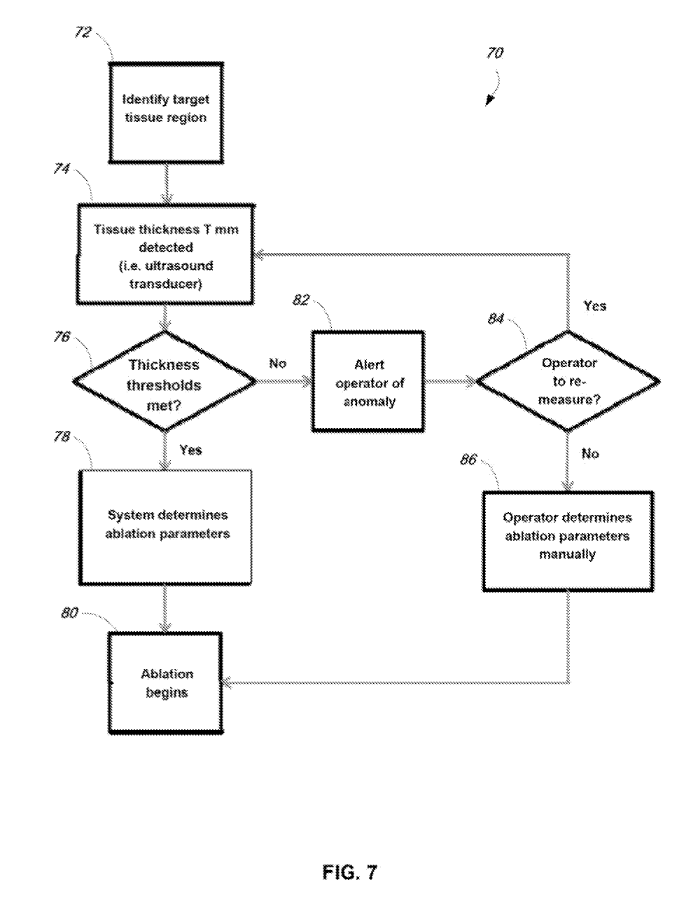

FIG. 7 shows a flowchart 70 with one example of a method for an algorithm that may be utilized, e.g., by a controller such as a microprocessor in communication with the ultrasound transducers 66, for controlling and/or adjusting various parameters of an ablation procedure, e.g., power, laser intensity, flow rate, temperature, etc. In this example, once the target tissue region has been identified 72 for treatment, such as visually or otherwise, the targeted tissue thickness may be detected 74, e.g., via the one or more ultrasound transducers 66. A tissue thickness threshold, e.g., a minimum tissue thickness, may be predetermined and programmed into the device for comparison against the detected thickness 76 to ensure patient safety.

In the event that the detected thickness fails to meet the threshold level, the operator may be alerted (visual or auditory) of this anomaly 82 prompting the operator to re-measure 84 the tissue thickness. If the re-measured tissue thickness meets the threshold level, the ablation procedure may continue. Otherwise, the operator may manually determine the ablation parameters 86, e.g., lowering power levels, etc., and begin the ablation procedure 80. In the event that the re-measured tissue thickness meets the threshold level 76, the controller may automatically determine the appropriate ablation parameters 78, e.g., based upon a table of ablation parameters for a given thickness value, and the ablation procedure may begin 80.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A tissue ablation apparatus, comprising:
an inflatable balloon attached to a catheter shaft and formed of an optically transparent membrane;
an optical fiber located within the balloon and positioned to direct optical radiation projecting from a distal end of the optical fiber to a distal end of the balloon; and,
an electronic imager affixed to an inner surface of the balloon and angled to provide a visual field of view of a distal end of the balloon.

2. The apparatus of claim 1 further comprising an outer membrane at least partially overlaid about the balloon such that an annular channel is formed between the outer membrane and balloon.

3. The apparatus of claim 2 wherein the outer membrane defines a plurality of pores near or at a distal end of the outer membrane.

4. The apparatus of claim 1 wherein the distal end of the optical fiber is articulatable to adjust a position of the distal end relative to the balloon.

5. The apparatus of claim 1 wherein the electronic imager comprises a CMOS or CCD imaging sensor.

6. The apparatus of claim 1 further comprising at least one light source located within the balloon.

7. The apparatus of claim 1 further comprising one or more electrodes positioned near or at a distal end of the balloon.

8. A tissue ablation apparatus, comprising:
an inflatable balloon attached to a catheter shaft and formed of an optically transparent membrane;
an optical fiber located within the balloon and positioned to direct optical radiation projecting from a distal end of the optical fiber to a distal end of the balloon; and,
an electronic imager located within the balloon and affixed to an articulatable member which is configurable to adjust its angle relative to the balloon.

9. A tissue ablation apparatus, comprising:
an inflatable balloon attached to a catheter shaft and formed of an optically transparent membrane;
an optical fiber located within the balloon and positioned to direct optical radiation projecting from a distal end of the optical fiber to a distal end of the balloon; and,
one or more electrodes positioned near or at a distal end of the balloon such that the electrodes contact or are in proximity to a region of tissue.

10. A method of treating a tissue region, comprising:
positioning an inflated balloon against a tissue region to be treated;
ablating the tissue region via radiative energy transmitted through a membrane of the balloon; and,
visualizing the tissue region through the membrane of the balloon via an imaging member positioned along an inner surface of the balloon.

11. The method of claim 10 wherein positioning comprises pressing the inflated balloon against the tissue region such that intervening fluid is displaced.

12. The method of claim 10 wherein ablating comprises transmitting laser energy from an optical fiber positioned within the balloon through the membrane and incident upon the tissue region.

13. The method of claim 10 wherein visualizing comprises illuminating the tissue region via at least one light source positioned within the balloon.

14. The method of claim 10 further comprising conducting electrical energy into the tissue region via a fluid in communication with one or more electrodes.

15. A method of treating a tissue region, comprising:
positioning an inflated balloon against a tissue region to be treated;
ablating the tissue region via radiative energy transmitted through a membrane of the balloon; and,
visualizing the tissue region through the membrane of the balloon via an imaging member affixed to an articulatable member which is configurable to adjust its angle relative to the balloon.

16. A method of treating a tissue region, comprising:
positioning an inflated balloon against a tissue region to be treated;
ablating the tissue region via radiative energy transmitted through a membrane of the balloon; and,
conducting electrical energy into the tissue region via a fluid in communication with one or more electrodes.
